(19) United States
(12) Patent Application Publication
(10) Pub. No.: US 2015/0086499 A1
(43) Pub. Date: Mar. 26, 2015

(54) THICKENED ANTIPERSPIRANT ROLL-ONS HAVING AN IMPROVED RESIDUE BEHAVIOR

(71) Applicant: Henkel AG & Co. KGaA, Duesseldorf (DE)

(72) Inventors: Thomas Doering, Dormagen (DE); Natascha Schevardo, Erkrath (DE)

(21) Appl. No.: 14/556,435

(22) Filed: Dec. 1, 2014

Related U.S. Application Data
(63) Continuation of application No. PCT/EP2013/061762, filed on Jun. 7, 2013.

(30) Foreign Application Priority Data
Jun. 8, 2012 (DE) 10 2012 209 621.3

(51) Int. Cl. A61K 8/73; A61K 8/28; A61K 8/26; A61Q 15/00

(52) U.S. Cl. CPC A61K 8/73; A61Q 15/00; A61K 8/28; A61K 8/26; A61K 8/731; A61K 2800/48; A61K 2800/592
USPC 424/66; 424/68

(57) ABSTRACT

Antiperspirant compositions that are suitable for application using a roll applicator include water, at least one perspiration-inhibiting aluminum compound or aluminum-zirconium compound, furthermore dehydroxanthan gum, and in addition thereto at least one nonionic thickening polymer selected from cellulose and cellulose ethers as well as mixtures thereof. The compositions exhibit elevated shelf stability even at relatively high temperatures, as well as reduced visible residua on the skin.

### FIELD OF THE INVENTION

The present invention generally relates to hydrous perspiration-inhibiting compositions, synonymously also referred to as “antiperspirant” compositions, which are suitable for application with a roll-on applicator and exhibit improved shelf stability even at relatively high temperatures, as well as reduced visible residue on the skin

### BACKGROUND OF THE INVENTION

There are numerous possibilities for applying perspiration-inhibiting compositions onto the skin. Dimensionally stable stick substances are spread over the skin from a stick dispenser until an effective quantity is deposited. Gels and creams can also be applied with stick-like dispensers, being spread over the skin using a dispenser surface. In particular for perspiration-inhibiting and/or deodorizing compositions for the underarm region, numerous different application forms besides those already recited have been developed, especially propellant-gas-containing and propellant-gas-free sprays and roll-on compositions. With the latter, a slightly thickened liquid is applied from a reservoir container via a rotatably mounted ball by rolling over the skin. Perspiration-inhibiting roll-on compositions can be anhydrous and oil-based; for example, the oil-based stock material of usual antiperspirant sprays is also suitable for administration as a roll-on. Here the perspiration-inhibiting active agent is present as a suspended powder in an oil that is thickened with a lipophilic gelling agent in order to prevent settling of the powder particles. Very few roll-ons of this kind are present on the market, however. Usual perspiration-inhibiting roll-on compositions are water-based, i.e. they include approx. 50 wt % and more of their total weight as water. The antiperspirant active agent, usually a perspiration-inhibiting aluminum compound or aluminum-zirconium compound, is present in dissolved form. Thickening is necessary here in order to enable applicability of the composition using a roll applicator. Thickening is usually accomplished with a hydrophilic thickening agent. In selecting the hydrophilic thickening agent, consideration must be given, for example, to the acidic pH of the roll-on composition; in other words, polyacrylates usually cannot be used for this. Instead, starches, starch derivatives, cellulose, and cellulose derivatives are utilized. These starches, starch derivatives, cellulose and cellulose derivatives can have an inherent difficulty in that they break down under the influence of the acidic antiperspirant active agents, so that the originally established viscosity of the composition decreases appreciably after only a short time, which in turn causes the composition runs off the skin after being rolled on and produces an unpleasant wet feel.

A further disadvantage of known antiperspirant roll-ons that are present as an emulsion is their insufficient temperature stability. This can result, for example in a context of large temperature fluctuations to which the products can be exposed during transport and storage, in coalescence of the droplets of the dispersed phase, which negatively affects product properties.

A further disadvantage of known water-based antiperspirant roll-ons is the “whitewash effect,” which occurs when the composition dries on the skin after application, and that portion of the antiperspirant salt which is not yet absorbed by the skin becomes visible as an undesired white residuum on the skin.

It is therefore desirable to provide water-based antiperspirant roll-ons having improved viscosity stability.

It is further desirable to provide antiperspirant roll-ons in the form of hydrous emulsions having improved temperature stability.

It is also desirable to provide water-based antiperspirant roll-ons having improved residuum behavior and, in particular, reduced white residue.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

It has been found, surprisingly, that the aforesaid objects are achieved by a combination of thickening agents, namely, dehydroxanthan gum in combination with at least one nonionic thickening polymer that is selected from cellulose and cellulose ethers as well as mixtures thereof.

Accordingly, the present invention is directed to an antiperspirant composition for roll-on application, including the following four components: water, at least one perspiration-inhibiting aluminum compound or aluminum-zirconium compound, dehydroxanthan gum, and at least one nonionic thickening polymer selected from cellulose and cellulose ethers as well as mixtures thereof.

### BRIEF DESCRIPTION OF THE DRAWINGS

The present invention will hereinafter be described in conjunction with the following drawing figures, wherein like numerals denote like elements, and

FIG. 1 shows the change in the viscosity of emulsions A and B, which were stored at a room temperature of 45° C., over a period of 8 weeks. After this time the non-inventive emulsion B had separated into an oil phase and water phase.

FIG. 2 shows the residua of 0.3 gram each of emulsions A (inventive) and C (not inventive) that were applied onto black paperboard and dried for 24 hours at 20° C. The inventive formula A left behind appreciably fewer visible residua than the comparison formula C.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present invention is therefore an antiperspirant composition for roll-on application, including the following four components:
a) water,
b) at least one perspiration-inhibiting aluminum compound or aluminum-zirconium compound,
c) dehydroxanthan gum,
d) at least one nonionic thickening polymer selected from cellulose and cellulose ethers as well as mixtures thereof.

Further preferred embodiments of the compositions according to the present invention may be gathered from the dependent claims.

### Water Content

The compositions according to the present invention include preferably 40 to 90 wt %, particularly preferably 50 to 85 wt %, extraordinarily preferably 60 to 80 wt %, more extraordinarily preferably 65 to 75 wt % water, based in each case on the total weight of the composition. For purposes of the present invention “water” means “free water,” i.e. water that is included in the antiperspirant composition not in the form of water of crystallization, water of hydration, or similarly molecularly bound water. The quantity of water of crystallization, water of hydration, or similarly molecularly bound water that is included in the constituents used, in particular in the perspiration-inhibiting active agents, does not represent free water for purposes of the present invention. Free water is that water which is included in the composition according to the present invention, for example, as a solvent or as a solvent constituent of other active agents.

“Standard conditions” for purposes of the present invention are a temperature of 20° C. (measurement reference temperature) and a pressure of 1013 mbar. Indications of melting point likewise refer to a pressure of 1013 mbar.

### Dehydroxanthan Gum

The compositions according to the present invention include dehydroxanthan gum preferably in a quantity from 0.05 to 1 wt %, particularly preferably 0.1 to 0.8 wt %, extraordinarily preferably 0.2 to 0.5 wt %, based in each case on the total weight of the composition.

The compositions according to the present invention furthermore include at least one nonionic thickening polymer that is selected from cellulose and cellulose ethers as well as mixtures thereof.

With the combination of dehydroxanthan gum and at least one nonionic thickening polymer that is selected from cellulose and cellulose ethers as well as mixtures thereof, the viscosity values of the compositions according to the present invention lie in the range between 1500 and 2500 mPas that is required in terms of application engineering, the viscosity being measured at 23° C. with a rotary viscometer of the Brookfield company, model RVF, spindle 4, shear rate (rotation frequency) 20 min⁻¹, no Helipath.

Water-based antiperspirant roll-ons that include only cellulose or a cellulose ether, for example hydroxyethyl cellulose, and no dehydroxanthan gum, exhibit unstable viscosity values as compared with the combination of dehydroxanthan gum and cellulose or cellulose ethers, for the same quantity of thickener. Water-based antiperspirant roll-ons that have been thickened only with dehydroxanthan gum are stable, but exhibit an undesired whitewash effect after application and drying (Example 1).

By combining dehydroxanthan gum with at least one nonionic thickening polymer that is selected from cellulose and cellulose ethers as well as mixtures thereof, perspiration-inhibiting oil-in-water emulsions having particularly favorable stability properties at temperatures of up to 45° C. are obtained. The viscosity values are in the range between 1500 and 2500 mPas (23° C.) that is required in terms of application engineering. Perspiration-inhibiting oil-in-water emulsions that include only the cellulose ether hydroxyethyl cellulose, and no dehydroxanthan gum, have already separated into two phases after eight weeks at 40° C.

Preferred cellulose ethers are selected from hydroxyalkyl celluloses, in particular from hydroxyethyl cellulose, hydroxypropyl cellulose, hydroxypropylmethyl cellulose, carboxymethyl cellulose, cetylhydroxyethyl cellulose, hydroxybutylmethyl cellulose, and methylhydroxyethyl cellulose, as well as mixtures thereof. Hydroxyethyl cellulose is extraordinarily preferred. It is also extraordinarily preferred that hydroxyethyl cellulose be included in the composition, alongside dehydroxanthan gum, as the only thickening polymer.

Preferred compositions according to the present invention include at least one nonionic thickening polymer that is selected from cellulose and cellulose ethers as well as mixtures thereof, in a total quantity from 0.05 to 1 wt %, preferably 0.1 to 0.7 wt %, particularly preferably 0.1 to 0.3 wt %, based in each case on the total weight of the composition.

Further preferred compositions include 0.05 to 1 wt %, preferably 0.1 to 0.7 wt %, particularly preferably 0.1 to 0.3 wt % hydroxyethyl cellulose, based in each case on the total weight of the composition.

Further preferred compositions according to the present invention contain, based in each case on the total weight of the composition, 0.05 to 1.0 wt %, preferably 0.1 to 0.8 wt %, particularly preferably 0.2 to 0.5 wt % dehydroxanthan gum and 0.05 to 1 wt %, preferably 0.1 to 0.7 wt %, particularly preferably 0.1 to 0.3 wt % hydroxyethyl cellulose.

Further preferred compositions according to the present invention are characterized in that dehydroxanthan gum and the total quantity of nonionic thickening polymer selected from cellulose and cellulose ethers as well as mixtures thereof are included at a weight ratio from 1 to 2.5, preferably 1.2 to 2.0, particularly preferably 1.4 to 1.6.

Further preferred compositions according to the present invention are characterized in that dehydroxanthan gum and hydroxyethyl cellulose are included at a weight ratio from 1 to 2.5, preferably 1.2 to 2.0, particularly preferably 1.4 to 1.6.

### Perspiration-Inhibiting Aluminum or Aluminum-Zirconium Compound

Particularly preferred antiperspirant active agents are selected from aluminum chlorohydrate, in particular aluminum chlorohydrate having the general formula [Al₂(OH)₅Cl.1-6 H₂O]ₙ, preferably [Al₂(OH)₅Cl.2-3 H₂O]ₙ, which can be present in nonactivated or in activated (depolymerized) form, as well as aluminum chlorohydrate having the general formula [Al₂(OH)₄Cl₂.1-6 H₂O]ₙ, preferably [Al₂(OH)₄Cl₂.2-3 H₂O]ₙ, which can be present in nonactivated or in activated (depolymerized) form.

The manufacture of preferred antiperspirant active agents is disclosed, for example, in U.S. Pat. No. 3,887,692, U.S. Pat. No. 3,904,741, U.S. Pat. No. 4,359,456, GB 2048229, and GB 1347950.

Also preferred are aluminum sesquichlorohydrate, aluminum dichlorohydrate, aluminum chlorohydrex propylene glycol (PG) or aluminum chlorohydrex polyethylene glycol (PEG), aluminum or aluminum-zirconium glycol complexes, e.g. aluminum or aluminum-zirconium propylene glycol complexes, aluminum sesquichlorohydrex PG or aluminum sesquichlorohydrex PEG, aluminum PG dichlorohydrex or aluminum PEG dichlorohydrex, aluminum hydroxide, furthermore selected from aluminum zirconium chlorohydrates, such as aluminum zirconium trichlorohydrate, aluminum zirconium tetrachlorohydrate, aluminum zirconium pentachlorohydrate, aluminum zirconium octachlorohydrate, aluminum zirconium chlorohydrate/glycine complexes, such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAl(SO₄)₂.12 H₂O, alum), dehydrated alum (KAl(SO₄)₂ having zero to 11 mol water of crystallization), aluminum undecylenoyl collagen amino acid, sodium aluminum lactate+aluminum sulfate, sodium aluminum chlorohydroxylactate, aluminum bromohydrate, aluminum chloride, aluminum salts of lipoamino acids, aluminum sulfate, aluminum lactate, aluminum chlorohydroxyallantoinate, and sodium aluminum chlorohydroxylactate, and mixtures thereof.

Antiperspirant active agents particularly preferred according to the present invention are selected from so-called “activated” aluminum and aluminum-zirconium salts, which are also referred to as “enhanced activity” antiperspirant active agents. Such active agents are known in the existing art and also commercially obtainable. Their manufacture is disclosed, for example, in GB 2048229, U.S. Pat. No. 4,775,528, and U.S. Pat. No. 6,010,688. Activated aluminum and aluminum-zirconium salts are generally produced by heat treatment of a relatively dilute solution of the salt (e.g. approximately 10 wt % salt), in order to increase its HPLC peak 4 to peak 3 area ratio. The activated salt can then be dried, in particular spray-dried, to a powder. Besides spray drying, drum drying is, for example, also suitable.

Activated aluminum and aluminum-zirconium salts typically have an HPLC peak 4 to peak 3 area ratio of at least 0.4, preferably at least 0.7, particularly preferably at least 0.9, wherein at least 70% of the aluminum is to be associated with these peaks.

Activated aluminum and aluminum-zirconium salts do not necessarily need to be used as a spray-dried powder. Perspiration-inhibiting active agents that are likewise preferred according to the present invention are nonaqueous solutions or solubilizates of an activated perspiration-inhibiting aluminum or aluminum-zirconium salt, for example in accordance with U.S. Pat. No. 6,010,688, which are stabilized against loss of activation (rapid decrease in the HPLC peak 4 to peak 3 area ratio) of the salt by the addition of an effective quantity of a polyvalent alcohol that comprises 3 to 6 carbon atoms and 3 to 6 hydroxyl groups, preferably propylene glycol, sorbitol, and pentaerythritol. Preferred compositions are, for example, those that contain, in wt % (USP): 18 to 45 wt % of an activated aluminum or aluminum-zirconium salt, 55 to 82 wt % of at least one anhydrous polyvalent alcohol having 3 to 6 carbon atoms and 3 to 6 hydroxyl groups, preferably propylene glycol, butylene glycol, diethylene glycol, dipropylene glycol, glycerol, sorbitol, and pentaerythritol, particularly preferably propylene glycol.

Also particularly preferred are complexes of activated perspiration-inhibiting aluminum or aluminum-zirconium salts with a polyvalent alcohol which include 20 to 50 wt %, particularly preferably 20 to 42 wt % activated perspiration-inhibiting aluminum or aluminum-zirconium salt and 2 to 16 wt % molecularly bound water, the remainder (to 100 wt %) being at least one polyvalent alcohol having 3 to 6 carbon atoms and 3 to 6 hydroxyl groups. Propylene glycol, propylene glycol/sorbitol mixtures, and propylene glycol/pentaerythritol mixtures are preferred alcohols of this kind. Complexes of this kind, preferred according to the present invention, of an activated perspiration-inhibiting aluminum or aluminum-zirconium salt with a polyvalent alcohol, are disclosed e.g. in U.S. Pat. No. 5,643,558 and U.S. Pat. No. 6,245,325.

Further preferred perspiration-inhibiting active agents are basic calcium-aluminum salts such as those disclosed e.g. in U.S. Pat. No. 2,571,030. These salts are manufactured by reacting calcium carbonate with aluminum chlorhydroxide or aluminum chloride and aluminum powder, or by adding calcium chloride dihydrate to aluminum chlorhydroxide.

Further preferred perspiration-inhibiting active agents are aluminum-zirconium complexes such as those disclosed e.g. in U.S. Pat. No. 4,017,599, which are buffered with salts of amino acids, in particular with alkali glycinates and alkaline-earth glycinates.

Further preferred perspiration-inhibiting active agents are activated aluminum or aluminum-zirconium salts such as those disclosed e.g. in U.S. Pat. No. 6,245,325 or U.S. Pat. No. 6,042,816, including 5 to 78 wt % (USP) of an activated perspiration-inhibiting aluminum or aluminum-zirconium salt, an amino acid or hydroxyalkanoic acid in a quantity such as to furnish a weight ratio of (amino acid or hydroxyalkanoic acid) to (Al+Zr) from 2:1 to 1:20, and preferably 1:1 to 1:10, as well as a water-soluble calcium salt in a quantity such as to furnish a Ca:(Al+Zr) weight ratio from 1:1 to 1:28, and preferably 1:2 to 1:25. Particularly preferred solid activated perspiration-inhibiting salt compositions, for example according to U.S. Pat. No. 6,245,325 or U.S. Pat. No. 6,042,816, include 48 to 78 wt % (USP), preferably 66 to 75 wt % of an activated aluminum or aluminum-zirconium salt and 1 to 16 wt %, preferably 4 to 13 wt % molecularly bound water (water of hydration), furthermore water-soluble calcium salt in a quantity sufficient that the Ca:(Al+Zr) weight ratio is 1:1 to 1:28, preferably 1:2 to 1:25, and sufficient amino acid that the (amino acid) to (Al+Zr) weight ratio is 2:1 to 1:20, preferably 1:1 to 1:10.

Further particularly preferred solid perspiration-inhibiting activated salt compositions, for example according to U.S. Pat. No. 6,245,325 or U.S. Pat. No. 6,042,816, include 48 to 78 wt % (USP), preferably 66 to 75 wt %, of an activated aluminum or aluminum-zirconium salt and 1 to 16 wt %, preferably 4 to 13 wt % molecularly bound water (water of hydration), furthermore water-soluble calcium salt in a quantity sufficient that the Ca:(Al+Zr) weight ratio is 1:1 to 1:28, preferably 1:2 to 1:25, and sufficient glycine that the (glycine) to (Al+Zr) weight ratio is 2:1 to 1:20, preferably 1:1 to 1:10.

Further particularly preferred solid perspiration-inhibiting activated salt compositions, for example according to U.S. Pat. No. 6,245,325 or U.S. Pat. No. 6,042,816, include 48 to 78 wt % (USP), preferably 66 to 75 wt % of an activated aluminum or aluminum-zirconium salt and 1 to 16 wt %, preferably 4 to 13 wt % molecularly bound water, furthermore water-soluble calcium salt in a quantity sufficient that the Ca:(Al+Zr) weight ratio is 1:1 to 1:28, preferably 1:2 to 1:25, and sufficient hydroxyalkanoic acid that the (hydroxyalkanoic acid) to (Al+Zr) weight ratio is 2:1 to 1:20, preferably 1:1 to 1:10.

Water-soluble calcium salts preferred for stabilization of the perspiration-inhibiting salts are selected from calcium chloride, calcium bromide, calcium nitrate, calcium citrate, calcium formate, calcium acetate, calcium gluconate, calcium ascorbate, calcium lactate, calcium glycinate, calcium carbonate, calcium sulfate, calcium hydroxide, and mixtures thereof.

Amino acids preferred for stabilization of the perspiration-inhibiting salts are selected from glycine, alanine, leucine, isoleucine, β-alanine, valine, cysteine, serine, tryptophan, phenylalanine, methionine, β-amino-n-butanoic acid, and γ-amino-n-butanoic acid and salts thereof, in each case in the d-form, the l-form, and the dl-form; glycine is particularly preferred.

Hydroxyalkanoic acids preferred for stabilization of the perspiration-inhibiting salts are selected from glycolic acid and lactic acid.

Further preferred activated aluminum salts are those of the general formula Al₂(OH)₆₋ₐXa, in which X is Cl, Br, I, or NO₃ and “a” is a value from 0.3 to 5, preferably from 0.8 to 2.5, and particularly preferably 1 to 2, so that the molar ratio Al:X is 0.9:1 to 2.1:1, as disclosed e.g. in U.S. Pat. No. 6,074,632. Some water of hydration is generally associatively bound into these salts, typically 1 to 6 mol water per mol salt. Aluminum chlorohydrate is particularly preferred (i.e. X is Cl in the formula above), and especially 5/6-basic aluminum chlorohydrate in which “a” is equal to 1, so that the molar ratio of aluminum to chlorine is 1.9:1 to 2.1:1.

Preferred activated aluminum-zirconium salts are those that represent mixtures or complexes of the above-described aluminum salts with zirconium salts of the formula ZrO(OH)_{2-pb}Y_{b}, where Y is Cl, Br, I, NO₃, or SO₄, b is a rational number from 0.8 to 2, and p is the valency of Y, as disclosed e.g. in U.S. Pat. No. 6,074,632. The zirconium salts as a rule also have some associatively bound water of hydration, typically 1 to 7 mol water per mol salt. The zirconium salt is preferably zirconyl hydroxychloride having the formula ZrO(OH)_{2-b-}Cl_{b}, in which b is a rational number from 0.8 to 2, preferably 1.0 to 1.9. Preferred aluminum-zirconium salts have an Al:Zr molar ratio from 2 to 10 and a metal:(X+Y) ratio from 0.73 to 2.1, preferably 0.9 to 1.5. A particularly preferred salt is aluminum zirconium chlorohydrate (i.e. X and Y are Cl), which has an Al:Zr ratio from 2 to 10 and a molar metal:Cl ratio from 0.9 to 2.1. The term “aluminum zirconium chlorohydrate” encompasses the tri-, tetra-, penta- and octachlorohydrate

Zirconium salts preferred according to the present invention have the general formula ZrO(OH)₂₋ₐClₐ.xH₂O where a=1.5 to 1.87 and x=1 to 7, and a and x are rational numbers. These zirconium salts are disclosed, for example, in the Belgian document BE 825146.

Further preferred perspiration-inhibiting active agents are disclosed in U.S. Pat. No. 6,663,854 and US 2004 0009133.

The perspiration-inhibiting active agents are present in dissolved form. The antiperspirant active agents can be used as non-aqueous solutions or as glycolic solubilizates.

Preferred aluminum-zirconium salts have a molar metal-to-chloride ratio from 0.9 to 1.3, preferably 0.9 to 1.1, particularly preferably 0.9 to 1.0.

Preferred aluminum zirconium chlorohydrates generally have the empirical formula AlₙZr(OH)_{[3n+4-m(n+1)]}(Cl)_{[m(n+1)]}, where n=2.0 to 10.0, preferably 3.0 to 8.0, m=0.77 to 1.11 (corresponding to a molar ratio of metal (Al+Zr) to chloride from 1.3 to 0.9), preferably m=0.91 to 1.11 (corresponding to M:Cl=1.1 to 0.9), and particularly preferably m=1.00 to 1.11 (corresponding to M:Cl=1.0 to 0.9), also very preferably m=1.02 to 1.11 (corresponding to M:Cl=0.98 to 0.9) and very preferably m=1.04 to 1.11 (corresponding to M:Cl=0.96 to 0.9).

Some water of hydration is generally associatively bound in these salts, typically 1 to 6 mol water per mol salt, corresponding 1 to 16 wt %, preferably 4 to 13 wt % water of hydration.

Preferred aluminum zirconium chlorohydrates are usually associated with an amino acid in order to prevent polymerization of the zirconium species during manufacture. Preferred stabilizing amino acids are selected from glycine, alanine, leucine, isoleucine, 3-alanine, cysteine, valine, serine, tryptophan, phenylalanine, methionine, β-amino-n-butanoic acid, and γ-amino-n-butanoic acid, and salts thereof, respectively in the d-form, l-form, and dl-form; glycine is particularly preferred. The amino acid is included in the salt in a quantity from 1 to 3 mol, preferably 1.3 to 1.8 mol, in each case per mol of zirconium.

Preferred perspiration-inhibiting salts are aluminum zirconium tetrachlorohydrate (Al:Zr=2 to 6; M:Cl=0.9 to 1.3), in particular salts having a molar metal-to-chloride ratio from 0.9 to 1.1, preferably 0.9 to 1.0.

Also preferred according to the present invention are aluminum zirconium chlorohydrate glycine salts that are stabilized with betaine ((CH₃)₃N⁺—CH₂—COO⁻). Particularly preferred corresponding compounds have a molar ratio of (total [betaine+glycine]) to Zr from (0.1 to 3.0):1, preferably (0.7 to 1.5):1, and a molar ratio of betaine to glycine of at least 0.001:1. Corresponding compounds are disclosed, for example, in U.S. Pat. No. 7,105,691.

In a particularly preferred embodiment according to the present invention, a so-called “activated” salt is included as a particularly effective antiperspirant salt, in particular one having a high HPLC peak 5 aluminum content, in particular having a peak 5 area of at least 33%, particularly preferably at least 45%, based on the total area under peaks 2 to 5, measured by HPLC of a 10 wt % aqueous solution of the active agent under conditions in which the aluminum species are resolved into at least four successive peaks (referred to as peaks 2 to 5). Preferred aluminum-zirconium salts having a high HPLC peak 5 aluminum content (also called “E⁵AZCH”) are disclosed, for example, in U.S. Pat. No. 6,436,381 and U.S. Pat. No. 6,649,152.

Also preferred are those activated “E⁵AZCH” salts whose HPLC peak 4 to peak 3 area ratio is equal to at least 0.4, preferably at least 0.7, particularly preferably at least 0.9.

Further particularly preferred antiperspirant active agents are those aluminum-zirconium salts having a high HPLC peak 5 aluminum content which are additionally stabilized with a water-soluble strontium salt and/or with a water-soluble calcium salt. Corresponding salts are disclosed, for example, in U.S. Pat. No. 6,923,952.

Particularly preferred compositions according to the present invention include at least one perspiration-inhibiting aluminum compound or aluminum-zirconium compound in a total quantity from 5 to 35 wt %, preferably 8 to 30 wt %, particularly preferably 10 to 25 wt %, and extraordinarily preferably 16 to 20 wt %, based in each case on the total weight of active substance (USP), free of water of crystallization and free of ligands, in the total composition.

Further particularly preferred compositions according to the present invention include at least one perspiration-inhibiting aluminum compound in a total quantity from 5 to 35 wt %, preferably 8 to 30 wt %, particularly preferably 10 to 25 wt %, and extraordinarily preferably 16 to 20 wt %, based in each case on the total weight of active substance (USP), free of water of crystallization and free of ligands, in the total composition.

Further particularly preferred compositions according to the present invention include at least one perspiration-inhibiting aluminum-zirconium compound in a total quantity from 5 to 35 wt %, preferably 8 to 30 wt %, particularly preferably 10 to 25 wt %, and extraordinarily preferably 16 to 20 wt %, based in each case on the total weight of active substance (USP), free of water of crystallization and free of ligands, in the total composition.

Preferred compositions according to the present invention include at least one oil-in-water emulsifier agent having an HLB value greater than 7 to 20, which is preferably selected from nonionic oil-in-water emulsifier agents having an HLB value from greater than 7 to 20.

These are emulsifier agents commonly known to one skilled in the art, as listed e.g. in Kirk-Othmer, “Encyclopedia of Chemical Technology”, 3rd ed., 1979, Vol. 8, pp. 913-916. For ethoxylated products the HLB value is calculated using the formula HLB=(100−L):5, where L is the weight proportion of the lipophilic groups, i.e. the fatty alkyl or fatty acyl groups, in the ethylene oxide adducts, expressed as percent by weight.

Further antiperspirant compositions preferred according to the present invention are characterized in that at least one nonionic emulsifier agent having an HLB agent in the range from 12 to 18 is included. Preferred antiperspirant compositions according to the present invention are characterized in that the nonionic oil-in-water emulsifier agents having an HLB value from greater than 7 to 20 are selected from ethoxylated C₈ to C₂₄ alkanols having an average of 10 to 100 mol ethylene oxide per mol, ethoxylated C₈ to C₂₄ carboxylic acids having an average of 10 to 100 mol ethylene oxide per mol, sorbitan monoesters, ethoxylated with an average of 20 to 100 mol ethylene oxide per mol, of linear saturated and unsaturated C₁₂ to C₂₄ carboxylic acids, which can be hydroxylated, in particular those of myristic acid, palmitic acid, stearic acid, or of mixtures of said fatty acids, silicone copolyols having ethylene oxide units or having ethylene oxide and propylene oxide units, alkylmono- and -oligoglycosides having 8 to 22 carbon atoms in the alkyl residue and ethoxylated analogs thereof, ethoxylated sterols, partial esters of polyglycerols having n=2 to 10 glycerol units and esterified with 1 to 4 saturated or unsaturated, linear or branched, optionally hydroxylated C₈ to C₃₀ fatty acid residues, provided they have an HLB value of more than 7 to 20, as well as mixtures of the substances recited above.

The ethoxylated C₈ to C₂₄ alkanols have the formula R¹OH(CH₂CH₂O)ₙH, where R¹ denotes a linear or branched alkyl residue and/or alkenyl residue having 8 to 24 carbon atoms, and n denotes the average number of ethylene oxide units per molecule, for numbers from 10 to 100, preferably 10 to 30 mol ethylene oxide per 1 mol caprylyl alcohol, 2-ethylhexyl alcohol, capryl alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, and brassidyl alcohol, as well as industrial mixtures thereof. Adducts of 10 to 100 mol ethylene oxide with industrial fatty alcohols having 12 to 18 carbon atoms, for example coconut, palm, palm kernel, or tallow fatty alcohol, are also suitable.

The ethoxylated C₈ to C₂₄ carboxylic acids have the formula R¹O(CH₂CH₂O)ₙOH, where R¹O denotes a linear or branched, saturated or unsaturated acyl residue having 8 to 24 carbon atoms and n denotes the average number of ethylene oxide units per molecule, for numbers from 10 to 100, preferably 10 to 30 mol ethylene oxide per 1 mol caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselic acid, arachidic acid, gadoleic acid, behenic acid, erucic acid, and brassidic acid, as well as technical mixtures thereof. Adducts of 10 to 100 mol ethylene oxide with industrial fatty acids having 12 to 18 carbon atoms, for example coconut, palm, palm kernel, or tallow fatty acid, are also suitable. PEG-50 monostearate, PEG-100 monostearate, PEG-50 monooleate, PEG-100 monooleate, PEG-50 monolaurate, and PEG-100 monolaurate are particularly preferred.

It is particularly preferred to use C₁₂ to C₁₈ alkanols or C₁₂ to C₁₈ carboxylic acids each having 10 to 30 units ethylene oxide per molecule, as well as mixtures of said substances, in particular Ceteth-10, Ceteth-12, Ceteth-20, Ceteth-30, Steareth-10, Steareth-12, Steareth-20, Steareth-21, Steareth-30, Ceteareth-10, Ceteareth-12, Ceteareth-20, Ceteareth-30, Laureth-12, and Beheneth-20.

Preferred sorbitan monoesters, ethoxylated with an average of 20 to 100 mol ethylene oxide per mol, of linear saturated and unsaturated C₁₂ to C₃₀ carboxylic acids, which can be hydroxylated, are selected from Polysorbate-20, Polysorbate-40, Polysorbate-60, and Polysorbate-80.

C₈ to C₂₂ alkyl mono- and oligoglycosides are also preferably used. C₈ to C₂₂ alkyl mono- and oligoglycosides represent known, commercially usual emulsifier agents. They are manufactured in particular by reacting glucose or oligosaccharides with primary alcohols having 8 to 22 carbon atoms. In terms of the glycoside residue, both monoglycosides, in which a cyclic sugar residue is glycosidically bound to the fatty alcohol, and oligomeric glycosides having a degree of oligomerization of up to approximately 8, preferably 1 to 2, are suitable. The degree of oligomerization is a statistical average that is based on a homolog distribution that is usual for technical products of this kind. Products that are obtainable under the Plantacare® trademark include a glucosidically bound C₈ to C₁₆ alkyl group on an oligoglucoside residue whose average degree of oligomerization is 1 to 2, in particular 1.2 to 1.4. Particularly preferred C₈ to C₂₂ alkyl mono- and oligoglycosides are selected from octyl glucoside, decyl glucoside, lauryl glucoside, palmityl glucoside, isostearyl glucoside, stearyl glucoside, arachidyl glucoside, and behenyl glucoside, as well as mixtures thereof. The acyl glucamides derived from glucamine are also suitable as nonionic oil-in-water emulsifier agents.

Ethoxylated sterols, in particular ethoxylated soy sterols, also represent oil-in-water emulsifiers suitable according to the present invention. The degree of ethoxylation must be greater than 5, preferably less than 10, in order to exhibit an HLB value greater than 7. Suitable commercial products are, for example, PEG-10 Soy Sterol, PEG-16 Soy Sterol, and PEG-25 Soy Sterol.

It is further preferred to use partial esters of polyglycerols having 2 to 10 glycerol units and esterified with 1 to 4 saturated or unsaturated, linear or branched, optionally hydroxylated C₈ to C₃₀ fatty acid esters, provided they have an HLB value from more than 7 to 20. Diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate, and decaglycerol trihydroxystearate are particularly preferred.

Particularly preferred antiperspirant compositions according to the present invention include at least one oil-in-water emulsifier agent having an HLB value from greater than 7 to 20 in a total quantity from 0.5 to 5 wt %, preferably 0.8 to 4 wt %, particularly preferably 1.2 to 3 wt %, and extraordinarily preferably 1.5 to 2 wt %, based in each case on the total composition.

Further antiperspirant compositions particularly preferred according to the present invention include at least one nonionic oil-in-water emulsifier agent having an HLB value in the range from 12 to 18 in a total quantity from 0.5 to 5 wt %, preferably 0.8 to 4 wt %, particularly preferably 1.2 to 3 wt %, and extraordinarily preferably 1.5 to 2 wt %, based in each case on the total composition.

Further antiperspirant compositions particularly preferred according to the present invention include at least one nonionic oil-in-water emulsifier agent having an HLB value in the range from 12 to 18 that is selected from linear saturated and unsaturated C₁₂ to C₂₄ alkanols that are etherified with 7 to 40 ethylene oxide units per molecule, in a total quantity from 0.5 to 5 wt %, preferably 0.8 to 4 wt %, particularly preferably 1.2 to 3 wt %, and extraordinarily preferably 1.5 to 2 wt %, based in each case on the total composition. The aforementioned oil-in-water emulsifier agents are selected particularly preferably from Steareth, Ceteth, Myristeth, Laureth, Trideceth, Arachideth, and Beheneth having respectively 7 to 40 ethylene oxide units per molecule, in particular Steareth-10, Steareth-20, Steareth-21, Steareth-30, Steareth-40, Ceteth-10, Ceteth-20, Ceteth-21, Ceteth-30, Ceteth-40, Laureth-10, Laureth-20, Laureth-30, Trideceth-10, Trideceth-20, and Trideceth-30, as well as mixtures thereof.

Further antiperspirant compositions preferred according to the present invention include at least one nonionic oil-in-water emulsifier agent having an HLB value in the range from 12 to 18 that is selected from Steareth-10, Steareth-20, Steareth-21, Steareth-30, Steareth-40, Ceteth-10, Ceteth-20, Ceteth-21, Ceteth-30, Ceteth-40, Laureth-10, Laureth-20, Laureth-30, Trideceth-10, Trideceth-20, and Trideceth-30, as well as mixtures thereof, in a total quantity from 0.5 to 5 wt %, preferably 0.8 to 4 wt %, particularly preferably 1.2 to 3 wt %, and extraordinarily preferably 1.5 to 2 wt %, based in each case on the total composition.

Further antiperspirant compositions preferred according to the present invention include at least one cosmetic oil and at least one oil-in-water emulsifier having an HLB value from greater than 7 to 20, and are present as an oil-in-water emulsion. For purposes of the present invention, the term “emulsion” does not cover microemulsions.

Particularly preferred antiperspirant compositions according to the present invention are present as an oil-in-water emulsion and include at least one oil-in-water emulsion having an HLB value from greater than 7 to 20 in a total quantity from 0.5 to 5 wt %, preferably 0.8 to 4 wt %, particularly preferably 1.2 to 3 wt %, and extraordinarily preferably 1.5 to 2 wt %, based in each case on the total composition.

Further antiperspirant compositions particularly preferred according to the present invention are present as an oil-in-water emulsion and include at least one oil-in-water emulsion having an HLB value in the range from 12 to 18 in a total quantity from 0.5 to 5 wt %, preferably 0.8 to 4 wt %, particularly preferably 1.2 to 3 wt %, and extraordinarily preferably 1.5 to 2 wt %, based in each case on the total composition.

Further antiperspirant compositions particularly preferred according to the present invention are present as an oil-in-water emulsion and include at least one nonionic oil-in-water emulsifier agent having an HLB value in the range from 12 to 18 that is selected from linear saturated and unsaturated C₁₂ to C₂₄ alkanols that are etherified with 7 to 40 ethylene oxide units per molecule, in a total quantity from 0.5 to 5 wt %, preferably 0.8 to 4 wt %, particularly preferably 1.2 to 3 wt %, and extraordinarily preferably 1.5 to 2 wt %, based in each case on the total composition. The aforementioned oil-in-water emulsifier agents are selected particularly preferably from Steareth, Ceteth, Myristeth, Laureth, Trideceth, Arachideth, and Beheneth having respectively 7 to 40 ethylene oxide units per molecule, in particular Steareth-10, Steareth-20, Steareth-21, Steareth-30, Steareth-40, Ceteth-10, Ceteth-20, Ceteth-21, Ceteth-30, Ceteth-40, Laureth-10, Laureth-20, Laureth-30, Trideceth-10, Trideceth-20, and Trideceth-30, as well as mixtures thereof.

Further antiperspirant compositions preferred according to the present invention are present as an oil-in-water emulsion and include at least one nonionic oil-in-water emulsifier agent having an HLB value in the range from 12 to 18 that is selected from Steareth-10, Steareth-20, Steareth-21, Steareth-30, Steareth-40, Ceteth-10, Ceteth-20, Ceteth-21, Ceteth-30, Ceteth-40, Laureth-10, Laureth-20, Laureth-30, Trideceth-10, Trideceth-20, and Trideceth-30, as well as mixtures thereof, in a total quantity from 0.5 to 5 wt %, preferably 0.8 to 4 wt %, particularly preferably 1.2 to 3 wt %, and extraordinarily preferably 1.5 to 2 wt %, based in each case on the total composition.

### Water-in-Oil Emulsifier Agents

Further antiperspirant compositions preferred according to the present invention include at least one water-in-oil emulsifier agent, preferably at least one nonionic water-in-oil emulsifier agent, having in each case an HLB value greater than 1.0 and less than or equal to 7.0, preferably in the range from 3 to 6. Some of these water-in-oil emulsifier agents are listed, for example, in Kirk-Othmer, “Encyclopedia of Chemical Technology,” 3rd ed. 1979, Vol. 8, page 913. As already mentioned, the HLB value can also be calculated for ethoxylated adducts.

The following are preferred as water-in-oil emulsifier agents:
linear or branched, saturated or unsaturated C₁₂ to C₃₀ alkanols that are respectively etherified with 1 to 4 ethylene oxide units per molecule, which extraordinarily preferably are selected from Steareth, Ceteth, Myristeth, Laureth, Trideceth, Arachideth, and Beheneth each having 1 to 4 ethylene oxide units per molecule, in particular Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3, and Trideceth-4, as well as mixtures thereof;
linear saturated alkanols having 12 to 30 carbon atoms, in particular having 16 to 22 carbon atoms, in particular cetyl alcohol, stearyl alcohol, arachidyl alcohol, behenyl alcohol, and lanolin alcohol, or mixtures of these alcohols such as those obtainable upon industrial hydrogenation of vegetable and animal fatty acids;
esters and, in particular, partial esters of a polyol having 2 to 6 carbon atoms and linear saturated and unsaturated fatty acids having 12 to 30, in particular 14 to 22 carbon atoms, which can be hydroxylated. Such esters or partial esters are, for example, mono- and diesters of glycerol or monoesters of propylene glycol with linear saturated and unsaturated C₁₂ to C₃₀ carboxylic acids, which can be hydroxylated, in particular those with palmitic and stearic acid, sorbitan mono-, di-, or triesters of linear saturated and unsaturated C₁₂ to C₃₀ carboxylic acids, which can be hydroxylated, in particular those of myristic acid, palmitic acid, stearic acid, or of mixtures of said fatty acids, pentaerythrityl mono-, tri-, and tetraesters and methylglucose mono- and diesters of linear saturated and unsaturated C₁₂ to C₃₀ carboxylic acids, which can be hydroxylated, of which the mono-, di-, tri-, and tetraesters of pentaerythritol with linear saturated fatty acids having 12 to 30, in particular 14 to 22 carbon atoms, which can be hydroxylated, as well as mixtures thereof, are particularly preferred. Mono- and diesters are particularly preferred according to the present invention. C₁₂ to C₃₀ fatty acid residues preferred according to the present invention are selected from lauric acid, myristic acid, palmitic acid, stearic acid, arachidic acid, and behenic acid residues; the stearic acid residue is particularly preferred. Nonionic water-in-oil emulsifier agents particularly preferred according to the present invention having an HLB value greater than 1.0 and less than or equal to 7.0 are selected from glyceryl monostearate, glyceryl distearate, glyceryl monopalmitate, glyceryl dipalmitate, and mixtures thereof;
sterols, i.e. steroids that carry a hydroxyl group on the C3 atom of the steroid skeleton and are isolated both from animal tissue (zoosterols, e.g. cholesterol, lanosterol) and from plants (phytosterols, e.g. ergosterol, stigmasterol, sitosterol) and from fungi and yeasts (mycosterols), and can have a low degree of ethoxylation (1 to 5 EO);
alkanols and carboxylic acids each having 8 to 24 carbon atoms, in particular having 16 to 22 carbon atoms, in the alkyl group and 1 to 4 ethylene oxide units per molecule, which have an HLB value greater than 1.0 and less than or equal to 7.0;
glycerol monoethers of saturated and/or unsaturated, branched and/or unbranched alcohols having a chain length from 8 to 30, in particular 12 to 18 carbon atoms,
partial esters of polyglycerols having n=2 to 10 glycerol units and esterified with 1 to 5 saturated or unsaturated, linear or branched, optionally hydroxylated C₈ to C₃₀ fatty acid esters, provided they have an HLB value greater than 1.0 to less than or equal to 7;
as well as mixtures of the aforesaid substances.

Particularly preferably the at least one water-in-oil emulsifier agent having an HLB value greater than 1.0 and less than or equal to 7.0, preferably in the range from 3 to 6, is selected from linear or branched, saturated or unsaturated C₁₂ to C₃₀ alkanols that respectively are etherified with 1 to 4 ethylene oxide units per molecule, which are selected extraordinarily preferably from Steareth, Ceteth, Myristeth, Laureth, Trideceth, Arachideth, and Beheneth having respectively 1 to 4 ethylene oxide units per molecule, in particular Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3, and Trideceth-4, as well as mixtures thereof.

It can be preferred according to the present invention to use only a single water-in-oil emulsifier. In another preferred embodiment the compositions according to the present invention include mixtures, in particular technical mixtures, of at least two water-in-oil emulsifiers.

Antiperspirant compositions preferred according to the present invention include at least one water-in-oil emulsifier agent having an HLB value greater than 1.0 and less than or equal to 7.0, preferably in the range from 3 to 6, in a total quantity from 1.8 to 3 wt %, preferably 2 to 2.8 wt %, and particularly preferably 2.4 to 2.6 wt %, based in each case on the total weight of the composition according to the present invention.

Further antiperspirant compositions preferred according to the present invention include at least one water-in-oil emulsifier agent having an HLB value in the range from 3 to 6 selected from Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3, and Trideceth-4, as well as mixtures thereof, in a total quantity from 1.8 to 3 wt %, preferably 2 to 2.8 wt %, and particularly preferably 2.4 to 2.6 wt %, based in each case on the total weight of the composition according to the present invention.

Further antiperspirant compositions preferred according to the present invention are present as an oil-in-water emulsion and include at least one water-in-oil emulsifier agent having an HLB value greater than 1.0 and less than or equal to 7.0, preferably in the range from 3 to 6, in a total quantity from 1.8 to 3 wt %, preferably 2 to 2.8 wt %, and particularly preferably 2.4 to 2.6 wt %, based in each case on the total weight of the composition according to the present invention.

Further antiperspirant compositions preferred according to the present invention are present as an oil-in-water emulsion and include at least one water-in-oil emulsifier agent having an HLB value in the range from 3 to 6, selected from Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3, and Trideceth-4, as well as mixtures thereof, in a total quantity from 1.8 to 3 wt %, preferably 2 to 2.8 wt %, and particularly preferably 2.4 to 2.6 wt %, based in each case on the total weight of the composition according to the present invention.

### Oils

Antiperspirant compositions preferred according to the present invention are present in the form of an oil-in-water emulsion and include at least one cosmetic oil, preferably in a total quantity from 0.1 to 15 wt %, particularly preferably 0.3 to 10 wt %, extraordinarily preferably 0.5 to 6 wt %, based in each case on the weight of the total antiperspirant composition according to the present invention.

A distinction is made in the context of cosmetic oils between volatile and nonvolatile oils. “Nonvolatile” oils are understood as those oils which, at 20° C. and an ambient pressure of 1013 hPa, have a vapor pressure of less than 2.66 Pa (0.02 mm Hg). “Volatile” oils are understood as those oils which, at 20° C. and an ambient pressure of 1013 hPa, have a vapor pressure from 2.66 Pa to 40,000 Pa (0.02 mm to 300 mm Hg), preferably 13 to 12,000 Pa (0.1 to 90 mm Hg), particularly preferably 15 to 3000 Pa, extraordinarily preferably 30 to 500 Pa.

Nonvolatile non-silicone oils particularly preferred according to the present invention are selected from the addition products of at least 6 ethylene-oxide and/or propylene-oxide units with mono- or polyvalent C₃₋₂₂ alkanols such as butanol, butanediol, myristyl alcohol, and stearyl alcohol, e.g. PPG-13-butyl ether, PPG-14 butyl ether, PPG-9 butyl ether, PPG-10 butanediol, PPG-15 stearyl ether, and mixtures thereof.

Compositions particularly preferred according to the present invention include at least one cosmetic oil selected from PPG-13 butyl ether, PPG-14 butyl ether, PPG-9 butyl ether, PPG-10 butanediol, PPG-15 stearyl ether, and mixtures thereof, in a total quantity from 0.1 to 15 wt %, particularly preferably 0.3 to 10 wt %, extraordinarily preferably 0.5 to 6 wt %, based on the weight of the total antiperspirant composition according to the present invention. Extraordinarily preferred compositions according to the present invention include 0.1 to 15 wt %, particularly preferably 0.3 to 10 wt %, extraordinarily preferably 0.5 to 6 wt % PPG-15 stearyl ether, based in each case on the weight of the total antiperspirant composition according to the present invention.

Further nonvolatile non-silicone oils particularly preferred according to the present invention are esters of linear or branched, saturated or unsaturated fatty alcohols having 2 to 30 carbon atoms with linear or branched, saturated or unsaturated fatty acids having 2 to 30 carbon atoms, which can be hydroxylated. Esters of linear or branched saturated fatty alcohols having 2 to 5 carbon atoms with linear or branched, saturated or unsaturated fatty acids having 10 to 18 carbon atoms, which can be hydroxylated, are preferred. Preferred examples thereof are isopropyl palmitate, isopropyl stearate, isopropyl myristate, 2-hexyldecyl stearate, 2-hexyldecyl laurate, isononyl isononanoate, 2-ethylhexyl palmitate, and 2-ethylhexyl stearate. Also preferred are isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid 2-butyl octanoate, diisotridecyl acetate, n-hexyl laurate, n-decyl oleate, oleyl oleate, oleyl erucate, erucyl oleate, C₁₂ to C₁₅ alkyl lactate, and di-C₁₂ to C₁₃ alkyl malate, as well as benzoic acid esters of linear or branched C₈₋₂₂ alkanols. Benzoic acid C₁₂ to C₁₅ alkyl esters are particularly preferred, obtainable e.g. as a commercial product Finsolv® TN (C₁₂ to C₁₅ alkyl benzoate), as well as benzoic acid isostearyl ester, obtainable e.g. as Finsolv® SB, 2-ethylhexyl benzoate, obtainable e.g. as Finsolv® EB, and benzoic acid 2-octyldodecyl ester, obtainable e.g. as Finsolv® BOD. Triethyl citrate is a further particularly preferred ester oil.

Further nonvolatile non-silicone oils preferred according to the present invention are selected from branched saturated or unsaturated fatty alcohols having 6 to 30 carbon atoms. These alcohols are often also referred to as “Guerbet alcohols,” since they are obtainable via the Guerbet reaction. Preferred alcohol oils are 2-hexyldecanol, 2-octyldodecanol, and 2-ethyhexyl alcohol. Isostearyl alcohol is likewise preferred. Further preferred nonvolatile oils are selected from mixtures of Guerbet alcohols and Guerbet alcohol esters, e.g. 2-hexyldecanol and 2-hexyldecyl laurate.

The term “triglyceride” used below means “glycerol triester.” Further nonvolatile oils preferred according to the present invention are selected from triglycerides of linear or branched, saturated or unsaturated, optionally hydroxylated C₈₋₃₀ fatty acids, provided they are liquid under standard conditions. The use of natural oils, e.g. soy oil, cottonseed oil, sunflower oil, palm oil, palm kernel oil, linseed oil, almond oil, castor oil, corn oil, rapeseed oil, olive oil, sesame oil, thistle oil, wheat germ oil, peach-kernel oil, and the liquid components of coconut oil and the like, can be particularly suitable. Synthetic triglyceride oils are particularly preferred, in particular Capric/Caprylic Triglycerides, e.g. the commercial products Myritol® 318 or Myritol® 331 (BASF) having unbranched fatty acid esters, as well as glyceryl triisostearin and glyceryl tri(2-ethylhexanoate) having branched fatty acid esters. Triglyceride oils of this kind preferably account for a proportion of less than 50 wt % of the total weight of all cosmetic oils in the composition according to the present invention.

Further nonvolatile non-silicone oils particularly preferred according to the present invention are selected from dicarboxylic acid esters of linear or branched C₂ to C₁₀ alkanols, in particular diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate.

Further nonvolatile non-silicone oils particularly preferred according to the present invention are selected from addition products of 1 to 5 propylene oxide units with mono- or polyvalent C₈₋₂₂ alkanols such as octanol, decanol, decanediol, lauryl alcohol, myristyl alcohol, and stearyl alcohol, preferably from PPG-2 myristyl ether and PPG-3 myristyl ether.

Further nonvolatile non-silicone oils particularly preferred according to the present invention are selected from symmetrical, asymmetrical, or cyclic esters of carbonic acid with C₆ to C₂₀ alcohols, e.g. di-n-caprylyl carbonate, or di-(2-ethylhexyl) carbonate. Esters of carbonic acid with C₁ to C₅ alcohols, however, e.g. glycerol carbonate or propylene carbonate, are not compounds suitable as a cosmetic oil.

Further oils that can be preferred according to the present invention are selected from esters of dimers of unsaturated C₁₂ to C₂₂ fatty acids (dimer fatty acids) with monovalent linear, branched, or cyclic C₂ to C₁₈ alkanols or with polyvalent linear or branched C₂ to C₆ alkanols. The total weight of dimer fatty acid esters is particularly preferably 0.5 to 10 wt %, preferably 1 to 5 wt %, based in each case on the total composition.

Volatile cosmetic oils are usually selected from among cyclic silicone oils having the INCI name Cyclomethicone. The INCI name Cyclomethicone is understood in particular to mean cyclotrisiloxane (hexamethylcyclotrisiloxane), cyclotetrasiloxane (octamethylcyclotetrasiloxane), cyclopentasiloxane (decamethylcyclopentasiloxane), and cyclohexasiloxane (dodecamethylcyclohexasiloxane). These oils have a vapor pressure of approx. 13 to 15 Pa at 20° C.

Cyclomethicones are known in the existing art as oils well suited for cosmetic products, in particular for perspiration-inhibiting and deodorizing compositions. Because of their persistence in the environment, however, it can be preferred according to the present invention to omit the use of cyclomethicones. In an especially preferred embodiment, the compositions according to the present invention include 0 to less than 1 wt % cyclomethicone, based on the weight of the composition.

A preferred cyclomethicone replacement substance is a mixture of C₁₃ to C₁₆ isoparaffins, C₁₂ to C₁₄ isoparaffins, and C₁₃ to C₁₅ alkanes, whose viscosity at 25° C. is in the range from 2 to 6 mPas and which has a vapor pressure at 20° C. in the range from 100 to 150 Pa. A mixture of this kind is obtainable, for example, from Presperse Inc. under the name SiClone SR-5.

Further preferred volatile silicone oils are selected from volatile linear silicone oils, in particular volatile linear silicone oils having 2 to 10 siloxane units, such as hexamethyldisiloxane (L₂), octamethyltrisiloxane (L₃), decamethyltetrasiloxane (L₄), as included e.g. in the commercial products DC 2-1184, Dow Corning® 200 (0.65 cSt), and Dow Corning® 200 (1.5 cSt) of Dow Corning, and low-molecular-weight Phenyl Trimethicone having a vapor pressure at 20° C. of approximately 2000 Pa, as obtainable e.g. from GE Bayer Silicones/Momentive under the name Baysilone Fluid PD 5.

Preferred antiperspirant compositions according to the present invention contain, because of the drier skin feel and faster active-agent release, at least one volatile silicone oil. Preferred volatile non-silicone oils are selected from C₈ to C₁₆ isoparaffins, in particular from isononane, isodecane, isoundecane, isododecane, isotridecane, isotetradecane, isopentadecane, and isohexadecane, as well as mixtures thereof. C₁₀ to C₁₃ isoparaffin mixtures, in particular those having a vapor pressure at 20° C. from 10 to 400 Pa, preferably 13 to 100 Pa, are preferred.

Further antiperspirant compositions preferred according to the present invention in the form of oil-in-water emulsions are characterized in that the at least one propylene glycol mono ester of branched saturated C₆ to C₃₀ alkanecarboxylic acids is selected from propylene glycol monoisostearate, propylene glycol monoisopalmitate, propylene glycol monoisobehenate, propylene glycol monoisoarachidate, propylene glycol monoisomyristate, propylene glycol monoisocaprate, propylene glycol monoisocaprinate, and propylene glycol monoisocaprylate, and mixtures thereof. Further antiperspirant compositions preferred according to the present invention in the form of oil-in-water emulsions are characterized in that the at least one branched saturated C₁₀ to C₃₀ alkanol is selected from isostearyl alcohol, isocetyl alcohol, isomyristyl alcohol, isotridecyl alcohol, isoarachidyl alcohol, isobehenyl alcohol, isocapryl alcohol, isocaprinyl alcohol, isocaprylyl alcohol, and mixtures thereof.

Further antiperspirant compositions preferred according to the present invention in the form of oil-in-water emulsions are characterized in that at least one nonionic emulsifier agent having an HLB value in the range from 3 to 6 and at least one nonionic emulsifier agent having an HLB value in the range from 12 to 18 are included.

Further antiperspirant compositions preferred according to the present invention in the form of oil-in-water emulsions are characterized in that at least one nonionic emulsifier agent having an HLB value in the range from 3 to 6 is included in a total quantity from 1.8 to 3 wt % and at least one nonionic emulsifier agent having an HLB value in the range from 12 to 18 is included in a total quantity from 1 to 2 wt %, the quantity indications being based in each case on the total weight of the composition according to the present invention.

Further antiperspirant compositions preferred according to the present invention in the form of oil-in-water emulsions are characterized in that Steareth-2 is included as a nonionic emulsifier agent having an HLB value in the range from 3 to 6, and simultaneously Steareth-21 is included as a nonionic emulsifier agent having an HLB value in the range from 12 to 18.

Further antiperspirant compositions preferred according to the present invention in the form of oil-in-water emulsions are characterized in that Steareth-2, Steareth-21, and PPG-15 stearyl ether are included.

Further antiperspirant compositions preferred according to the present invention include in total a maximum of 3 wt %, preferably a maximum of 1 wt %, and particularly preferably 0 wt % monovalent C₁ to C₃ alkanols, such as ethanol or propanol, based in each case on the total weight of the composition according to the present invention.

### Additives

In addition to the ingredients recited above, the compositions according to the present invention can include further adjuvants and additives that, for example, improve their shelf life, such as preservatives, e.g. phenoxyethanol, methylparaben, or propylparaben, antioxidants, e.g. tetradibutyl pentaerythrityl hydroxyhydrocinnamate, liphochroman-6, tocopherol, tocopheryl acetate, or ascorbic acid and derivatives thereof, vitamins and derivatives thereof, such as tocopherol, tocopheryl acetate, ascorbic acid, panthenol, or pantolactone, perfumes, essential oils, menthol and menthol derivatives that exhibit a skin-cooling effect, care-providing substances having a skin-soothing effect, such as bisabolol and allantoin, active agents that delay hair growth, e.g. elformithine or glycyrrhizin and derivatives of the latter, moisturizers and humectants such as 1,2-propylene glycol, glycerol, 2-methyl-1,3-propanediol, 1,2-butylene glycol, 1,3-butylene glycol, 1,4-butylene glycol, pentylene glycols such as 1,2-pentanediol and 1,5-pentanediol, hexanediols such as 1,2-hexanediol and 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomer mixtures of cis- and trans-1,4-dimethylolcyclohexane, urea, N,N′-bis-(2-hydroxyethyl)urea, sodium pyrrolidone carboxylate, plant extracts, e.g. aloe vera extract, natural fats and oils such as jojoba oil, evening primrose oil, or linseed oil, saturated and unsaturated fatty acids such as stearic acid, oleic acid, linoleic acid, linolenic acid, or gamma-linolenic acid, squalane, squalene, deodorant active agents such as silver salts, colloidal silver, zeolites, 2-benzylheptan-1-ol, anisyl alcohol, mixtures of 2-benzylheptan-1-ol and phenoxyethanol, 3-(2-ethylhexyloxy)-1,2-propanediol, or tropolone, as well as mixtures of these substances.

A further subject of the present invention is the use of a combination of dehydroxanthan gum and at least one nonionic thickening polymer selected from cellulose and cellulose ethers as well as mixtures thereof to reduce residua and/or to improve the viscosity stability of an antiperspirant composition for roll-on application that includes water and at least one perspiration-inhibiting aluminum compound or aluminum-zirconium compound and optionally comprises the characterizing feature in accordance with at least one of claims 2 to 14.

The exemplifying embodiments below are intended to elucidate the subject matter of the present invention without limiting it thereto.

| | Formula A | Formula B |
|---|---|---|
| | (wt %) | (wt %) |
| Steareth-21 | 1.48 | 1.48 |
| Steareth-2 | 2.38 | 2.38 |
| PPG-15 stearyl ether | 0.50 | 0.50 |
| Water, demineralized | 54.09 | 54.09 |
| Aluminum chlorohydrate 50% | 40.00 | 40.00 |
| Phenoxyethanol | 0.90 | 0.90 |
| Amaze XT | 0.40 | — |
| Tylose H 100000 YP2 | 0.25 | 0.65 |
| Sodium hydroxide pearls | 0.01 | 0.01 |
| | Formula A | Formula C |
| | (wt %) | (wt %) |
| Steareth-21 | 1.48 | 1.48 |
| Steareth-2 | 2.38 | 2.38 |
| PPG-15 stearyl ether | 0.50 | 0.50 |
| Water, demineralized | 54.09 | 54.09 |
| Aluminum chlorohydrate 50% | 40.00 | 40.00 |
| Phenoxyethanol | 0.90 | 0.90 |
| Amaze XT | 0.40 | 0.65 |
| Tylose H 100000 YP2 | 0.25 | — |
| Sodium hydroxide pearls | 0.01 | 0.01 |

Tylose H 100000 YP2 = Hydroxyethyl cellulose (ex Shin-Etsu)
Amaze XT = dehydroxanthan gum (ex Akzo Nobel)

Formulations A (inventive), B (not inventive), and C (not inventive) represent perspiration-inhibiting oil-in-water emulsions (not microemulsions).

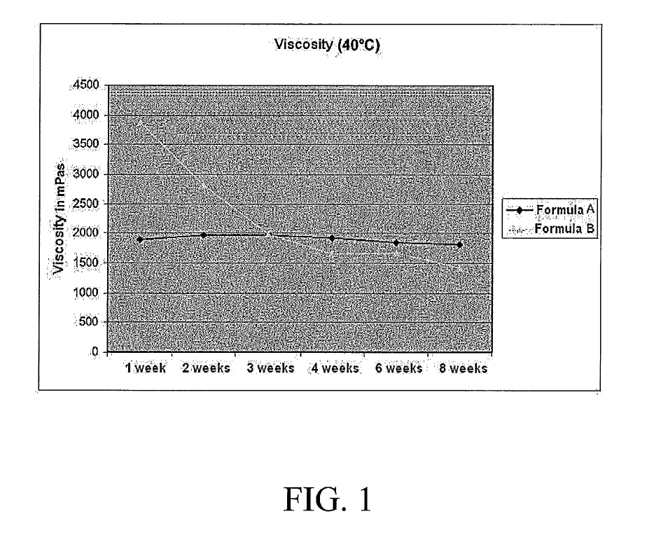

FIG. 1 shows the change in the viscosity of emulsions A and B, which were stored at a room temperature of 45° C., over a period of 8 weeks. After this time the non-inventive emulsion B had separated into an oil phase and water phase.

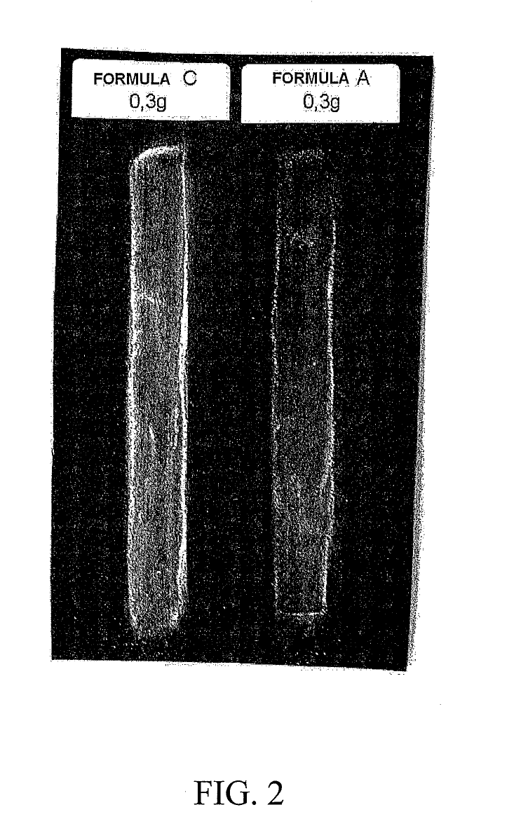

FIG. 2 shows the residua of 0.3 gram each of emulsions A (inventive) and C (not inventive) that were applied onto black paperboard and dried for 24 hours at 20° C. The inventive formula A left behind appreciably fewer visible residua than the comparison formula C.

Some preferred compositions according to the present invention can be gathered from the tables below (all quantity indications are in wt % based on the total weight of the composition):

| | 1 | 2 | 3 | 4 |
|---|---|---|---|---|
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Total qty. of at least one | 0.05-1 | 0.1-0.7 | 0.1-0.3 | 0.1-0.3 |
| nonionic thickening polymer | | | | |
| selected from cellulose, | | | | |
| cellulose ethers, and mixtures | | | | |
| thereof | | | | |
| | 5 | 6 | 7 | 8 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Total qty. of at least one | 0.05-1 | 0.1-0.7 | 0.1-0.3 | 0.1-0.3 |
| cellulose ether | | | | |
| | 9 | 10 | 11 | 12 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Hydroxyethyl cellulose | 0.05-1 | 0.1-0.7 | 0.1-0.3 | 0.1-0.3 |
| | 13 | 14 | 15 | 16 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Total qty. of at least one | 0.05-1 | 0.1-0.7 | 0.1-0.3 | 0.1-0.3 |
| cellulose ether | | | | |
| Total qty. of at least one oil-in- | 0.5-5 | 0.8-4 | 1.2-3 | 1.5-2 |
| water emulsifier agent having an | | | | |
| HLB value from >7 to 20 | | | | |
| | 17 | 18 | 19 | 20 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Hydroxyethyl cellulose | 0.05-1 | 0.1-0.7 | 0.1-0.3 | 0.1-0.3 |
| Total qty. of at least one oil-in- | 0.5-5 | 0.8-4 | 1.2-3 | 1.5-2 |
| water emulsifier agent having an | | | | |
| HLB value from >7 to 20 | | | | |
| | 21 | 22 | 23 | 24 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Total qty. of at least one | 0.05-1 | 0.1-0.7 | 0.1-0.3 | 0.1-0.3 |
| cellulose ether | | | | |
| Total qty. of at least one oil-in- | 0.5-5 | 0.8-4 | 1.2-3 | 1.5-2 |
| water emulsifier agent having an | | | | |
| HLB value from >7 to 20 | | | | |
| | 25 | 26 | 27 | 28 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Hydroxyethyl cellulose | 0.05-1 | 0.1-0.7 | 0.1-0.3 | 0.1-0.3 |
| Total qty. of at least one oil-in- | 0.5-5 | 0.8-4 | 1.2-3 | 1.5-2 |
| water emulsifier agent having an | | | | |
| HLB value from >7 to 20 | | | | |
| | 29 | 30 | 31 | 32 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Total qty. of at least one | 0.05-1 | 0.1-0.7 | 0.1-0.3 | 0.1-0.3 |
| cellulose ether | | | | |
| Steareth-21 | 0.5-5 | 0.8-4 | 1.2-3 | 1.5-2 |
| | 33 | 34 | 35 | 36 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Hydroxyethyl cellulose | 0.05-1 | 0.1-0.7 | 0.1-0.3 | 0.1-0.3 |
| Steareth-21 | 0.5-5 | 0.8-4 | 1.2-3 | 1.5-2 |

| | 37 | 38 | 39 | 40 |
|---|---|---|---|---|
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Total qty. of at least one | 0.05-1 | 0.1-0.7 | 0.1-0.3 | 0.1-0.3 |
| cellulose ether | | | | |
| Total qty. of at least one oil-in- | 0.5-5 | 0.8-4 | 1.2-3 | 1.5-2 |
| water emulsifier agent having an | | | | |
| HLB value from >7 to 20 | | | | |
| Total qty. of at least one water- | 1.8-3 | 2-2.8 | 2-2.8 | 2.4-2.6 |
| in-oil emulsifier agent having an | | | | |
| HLB value >1.0 and ≦7.0 | | | | |
| | 41 | 42 | 43 | 44 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Hydroxyethyl cellulose | 0.05-1 | 0.1-0.7 | 0.1-0.3 | 0.1-0.3 |
| Total qty. of at least one oil-in- | 0.5-5 | 0.8-4 | 1.2-3 | 1.5-2 |
| water emulsifier agent having an | | | | |
| HLB value from >7 to 20 | | | | |
| Total qty. of at least one water- | 1.8-3 | 2-2.8 | 2-2.8 | 2.4-2.6 |
| in-oil emulsifier agent having an | | | | |
| HLB value >1 .0 and ≦7.0 | | | | |
| | 45 | 46 | 47 | 48 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Total qty. of at least one | 0.05-1 | 0.1-0.7 | 0.1-0.3 | 0.1-0.3 |
| cellulose ether | | | | |
| Total qty. of at least one oil-in- | 0.5-5 | 0.8-4 | 1.2-3 | 1.5-2 |
| water emulsifier agent having an | | | | |
| HLB value from >7 to 20 | | | | |
| Total qty. of at least one water- | 1.8-3 | 2-2.8 | 2-2.8 | 2.4-2.6 |
| in-oil emulsifier agent having an | | | | |
| HLB value >1.0 and ≦7.0 | | | | |
| Total qty. of at least one | 0.1-15 | 0.3-10 | 0.5-6 | 0.5-6 |
| cosmetic oil | | | | |
| | 49 | 50 | 51 | 52 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Hydroxyethyl cellulose | 0.05-1 | 0.1-0.7 | 0.1-0.3 | 0.1-0.3 |
| Total qty. of at least one oil-in- | 0.5-5 | 0.8-4 | 1.2-3 | 1.5-2 |
| water emulsifier agent having an | | | | |
| HLB value from >7 to 20 | | | | |
| Total qty. of at least one water- | 1.8-3 | 2-2.8 | 2-2.8 | 2.4-2.6 |
| in-oil emulsifier agent having an | | | | |
| HLB value >1.0 and ≦7.0 | | | | |
| Total qty. of at least one | 0.1-15 | 0.3-10 | 0.5-6 | 0.5-6 |
| cosmetic oil | | | | |
| | 53 | 54 | 55 | 56 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Total qty. of at least one | 0.05-1 | 0.1-0.7 | 0.1-0.3 | 0.1-0.3 |
| cellulose ether | | | | |
| Total qty. of at least one | 0.5-5 | 0.8-4 | 1.2-3 | 1.5-2 |
| nonionic oil-in-water emulsifier | | | | |
| agent having an HLB value | | | | |
| from >7 to 20 | | | | |
| Total qty. of at least one | 1.8-3 | 2-2.8 | 2-2.8 | 2.4-2.6 |
| nonionic water-in-oil emulsifier | | | | |
| agent having an HLB value >1 .0 | | | | |
| and ≦7.0 | | | | |
| Total qty. of at least one | 0.1-15 | 0.3-10 | 0.5-6 | 0.5-6 |
| cosmetic oil | | | | |
| | 57 | 58 | 59 | 60 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Hydroxyethyl cellulose | 0.05-1 | 0.1-0.7 | 0.1-0.3 | 0.1-0.3 |
| Total qty. of at least one | 0.5-5 | 0.8-4 | 1.2-3 | 1.5-2 |
| nonionic oil-in-water emulsifier | | | | |
| agent having an HLB value | | | | |
| from >7 to 20 | | | | |
| Total qty. of at least one | 1.8-3 | 2-2.8 | 2-2.8 | 2.4-2.6 |
| nonionic oil-in-water emulsifier | | | | |
| agent having an HLB value >1.0 | | | | |
| and ≦7.0 | | | | |
| Total qty. of at least one | 0.1-15 | 0.3-10 | 0.5-6 | 0.5-6 |
| cosmetic oil | | | | |
| | 61 | 62 | 63 | 64 |
| Water | 40-90 | 50-85 | 60-80 | 65-75 |
| Total qty. of at least one | 5-35 | 8-30 | 10-25 | 16-20 |
| perspiration-inhibiting Al | | | | |
| compound or Al—Zr compound | | | | |
| Dehydroxanthan gum | 0.05-1 | 0.1-0.8 | 0.2-0.5 | 0.2-0.5 |
| Hydroxyethyl cellulose | 0.05-1 | 0.1-0.7 | 0.1-0.3 | 0.1-0.3 |
| Steareth-21 | 0.5-5 | 0.8-4 | 1.2-3 | 1.5-2 |
| Steareth-2 | 1.8-3 | 2-2.8 | 2-2.8 | 2.4-2.6 |
| PPG-15 stearyl ether | 0.1-15 | 0.3-10 | 0.5-6 | 0.5-6 |

The inventive compositions 45 to 64 are present in the form of an oil-in-water emulsion.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant composition for roll-on application, comprising:
a) water,
b) at least one perspiration-inhibiting aluminum compound or aluminum-zirconium compound,
c) dehydroxanthan gum, and
d) at least one nonionic thickening polymer selected from cellulose and cellulose ethers as well as mixtures thereof.

2. The composition according to claim 1, wherein the dehydroxanthan gum is included at a concentration ranging between 0.05 and 1 wt %, based on the total weight of the composition.

3. The composition according to claim 1, wherein the at least one nonionic thickening polymer that is selected from cellulose and cellulose ethers as well as mixtures thereof is included at a concentration ranging between 0.05 and 1 wt %, based on the total weight of the composition.

4. The composition according to claim 1, wherein the dehydroxanthan gum and the total quantity of nonionic thickening polymer selected from cellulose and cellulose ethers and mixtures thereof are included at a weight ratio from 1 to 2.5.

5. The composition according to claim 1, wherein the nonionic thickening polymer comprises hydroxyethyl cellulose.

6. The composition according to claim 1, wherein the dehydroxantham gum is included a concentration ranging between 0.1 and 0.8 wt %, and the nonionic thickening polymer comprises hydroxyethyl cellulose at a concentration ranging between 0.1 and 0.7 wt %, based in each case on the total weight of the composition.

7. The composition according to claim 1, further comprising at least one nonionic oil-in-water emulsifier agent having an HLB value from more than 7 to 20.

8. The composition according to claim 1, further comprising at least one water-in-oil emulsifier agent having an HLB value greater than 1.0 and less than or equal to 7.0.

9. The composition according to claim 1, further comprising at least one cosmetic oil in a total quantity from 0.1 to 15 wt % based on the weight of the total antiperspirant composition.

10. The composition according to claim 1, further comprising at least one cosmetic oil and at least one oil-in-water emulsifier agent having an HLB value from more than 7 to 20, wherein the antiperspirant composition is constituted as an oil-in-water emulsion that does not represent a microemulsion.

11. The composition according to claim 1, further comprising at least one cosmetic oil, at least one oil-in-water emulsifier agent having an HLB value from more than 7 to 20, and at least one water-in-oil emulsifier agent having an HLB value greater than 1.0 and less than or equal to 7.0, wherein the antiperspirant composition is constituted as an oil-in-water emulsion that does not represent a microemulsion.

12. The composition according to claim 1, further comprising at least one cosmetic oil in a total quantity from 0.1 to 15 wt %, at least one oil-in-water emulsifier agent having an HLB value from more than 7 to 20 in a total quantity from 0.5 to 5 wt %, and at least one water-in-oil emulsifier agent having an HLB value greater than 1.0 and less than or equal to 7.0 in a total quantity from 1.8 to 3 wt %, based in each case on the weight of the total antiperspirant composition, wherein the antiperspirant composition is constituted as an oil-in-water emulsion that does not represent a microemulsion.

13. The composition according to claim 1, further comprising at least one cosmetic oil in a total quantity from 0.5 to 6 wt %, at least one oil-in-water emulsifier agent having an HLB value from more than 7 to 20 in a total quantity from 1.2 to 3 wt %, and at least one water-in-oil emulsifier agent having an HLB value greater than 1.0 and less than or equal to 7.0 in a total quantity from 1.8 to 3 wt %, based in each case on the weight of the total antiperspirant composition according to the present invention, wherein the antiperspirant composition is constituted as an oil-in-water emulsion that does not represent a microemulsion.

14. The composition according to claim 1, wherein the dehydroxanthum gum is included at a concentration ranging between 0.1 and 0.8 wt % and the nonionic thickening polymer comprises hydroxyethyl cellulose at a concentration ranging between 0.1 and 0.7 wt %, and further wherein the composition comprises at least one cosmetic oil in a total quantity from 0.5 to 6 wt %, at least one oil-in-water emulsifier agent having an HLB value from more than 7 to 20 in a total quantity from 1.2 to 3 wt %, and at least one water-in-oil emulsifier agent having an HLB value greater than 1.0 and less than or equal to 7.0 in a total quantity from 1.8 to 3 wt %, based in each case on the weight of the total antiperspirant composition, and further wherein the composition is constituted as an oil-in-water emulsion that does not represent a microemulsion.
